(19) United States
(12) Patent Application Publication
(10) Pub. No.: US 2004/0186087 A1
(43) Pub. Date: Sep. 23, 2004

(54) Siderophore conjugates of photoactive dyes for photodynamic therapy

(75) Inventors: Susanna Grafe, Jena (DE); Peter Gebhardt, Jena (DE); Volker Albrecht, Jena (DE)

Correspondence Address: BOLESH J. SKUTNIK PhD.JD, 515 Shaker Road, East Longmeadow, MA, 01028, US

(73) Assignee: CeramOptec Industries, Inc.

(21) Appl. No.: 10/392,557

(22) Filed: Mar. 20, 2003

(51) Int. Cl. A61K 31/555; C07D 487/22; A61K 31/409

(52) U.S. Cl. 514/185; 514/410; 514/6; 540/145

(57) ABSTRACT

Siderophore-photosensitizer conjugates, their synthesis and use in photodynamic antimicrobial therapy (PACT) is disclosed. The advantage of this method is improvement of photodynamic antimicrobial therapy against, for example, pathogenic micro-organisms such as bacteria and fungi. Naturally occurring and synthetically available siderophore structures are conjugated chemically with photoactive compounds such as Chlorin e₆ to improve their penetration into bacterial cells and to increase antibacterial efficacy of photosensitizers via microbial proteins that recognize and transport iron-loaded siderophores. In this way, photosensitizers can be transported inside bacteria that otherwise could not cross the cell wall and membranes. Photodynamic activation of photosensitizers inside the cells of pathogenic microbes enables a more effective inhibition of cellular functions than application at the outer side of the cells. The siderophore-transporting systems of microbes are known to be specific for bacteria and fungi. Consequently, siderophore conjugates with photosensitizers are not taken up by mammalian cells and photodynamic effects can thus be exerted specifically on pathogenic microbes. Applications of these conjugates include highly efficient treatment of pathogenic gram-negative and -positive bacteria such as Pseudomonas aeruginosa, Escherichia coli, Streptococcus pyogenes, Staphylococcus aureus, treatment of microbial infections that often occur in chronic wounds as well as therapy of other antibiotic resistant microbial infections.

### BACKGROUND OF THE INVENTION

1. Field of the Invention

The present invention concerns the synthesis and usage of novel “siderophore-photosensitizer conjugates” in the photodynamic antimicrobial therapy.

2. Information Disclosure Statement

Photodynamic therapy (PDT) is one of the most promising new techniques being explored for use m a variety of medical applications and is known as a well-recognized treatment for the destruction of tumors (T.Okunara, H. Kato, Rev. Contemp. Pharmacother. 10 (1999) pp.59-68; “photodynamic therapeutics: basic principles and clinical applications”, W. M. Sharman, C. M. Allen, J. E. van Lier, DDT, 4 (1999) 507-517; “Pharmaceutical development and medical applications of porphyrin-type macromolecules”, E. D. Sternberg, D. Dolphin, C. Brueckner, Tetrahedron, 54 (1998) 4151-4202). Another important application of PDT is the treatment of infectious diseases due to pathogenic micro organisms including dermal, dental, suppurative, respiratory, gastro enteric, genital and other infections.

A constant problem in the treatment of infectious disease is the lack of specificity of the agents used for the treatment of disease, which results in the patient gaining a new set of maladies from the therapy.

The use of PDT for the treatment of various types of disease is limited due to the inherent features of photosensitizers. These include their high cost, extended retention in the host organism, substantial skin photo toxicity, background toxicity, low solubility in physiological solutions (which reduces its usefulness for intravascular administration as it can provoke thromboembolic accidents), and low targeting effectiveness. These disadvantages lead to the administration of extremely high doses of a photosensitizer, which dramatically increase the possibility of accumulation of the photosensitizer in non-damaged tissues and the accompanying risk of affecting non-damaged sites.

One of the prospective approaches to increase the specificity of photosensitizers and the effectiveness of PDT is a conjugation of a photosensitizer with a ligand-vector, which specifically binds to receptors on the surface of a target cell. A number of natural and synthetic molecules recognized by target cells can be used as such vectors. This approach is now used in the design of new generations of photosensitizers for the treatment of tumors (“Porphyrin-based photosensitizers for use in photodynamic therapy” E. D. Stemberg, D. Dolphin, C. Brueckner, Tetrahedron, 54 (1998) 4151-4202).

A number of problems remain in the use of PDT as an anti-microbial treatment. One such problem is the antibiotic resistance of Gram-negative bacterial pathogens as well as gram positive bacteria, due to the limited permeability of the outer membrane, which hampers an effective antimicrobial therapy.

Various therapeutic molecules have been conjugated with siderophores, iron-chelating ligands used by bacteria to scavenge ionic iron from the environment, to aid in targeting such molecules. Iron is one of the most abundant elements on earth. However, under physiological conditions, most commonly occurring ionic forms of iron are very weakly soluble in water and, consequently, there is a very low concentration of free iron (III) ions in nature. In order to scavenge low amounts of iron from the medium, many microbes, including pathogenic bacteria such as Pseudomonas aeruginosa, Escherichia coli and Salmonella typhimurium and fungi, produce and utilize very specific low molecular weight iron chelators known as Siderophores.

At physiological pH, free [Fe³⁺] concentration is limited to 10⁻¹⁸ M, whereas all living micro organisms require a minimum effective concentration of 10⁻⁸ M for growth. This limitation of an essential microbial nutrient is overcome by synthesizing and excreting siderophores as iron chelators and transporting vehicles. Thereby in the Gram-negative bacteria, the ferric ion-siderophore complex must cross the outer membrane and the cytoplasmatic membrane for the deliverance of iron into the cytoplasm. Since ferric complexes are unsuitable for passive diffusion or non-specific transport across these membranes their uptake is mediated by receptor enabling an active energy-dependent transport from outside to inside of microbial cells. The binding and transport of a ferric-siderophore to its receptor is usually highly specific (Stintzi A, Barnes C, Xu J and Raymond K N; PNAS 2000, Vol. 97, No. 20, 10691-10696).

Chemically, the siderophores mostly commonly contain catecholate or hydroxamate groups as iron-chelating ligands which are connected to peptides or oligoesters as scaffolds. Examples for bacterial siderophores are Enterobactin as a trimer of N-(2,3-dihydroxabenzoyl)-serine from E. coli Pyoverdin, found in P. aeruginosa and N-(2,3-dihydroxybenzoyl)-glycine from Bacillus subtilis.

Anti-microbial therapy is frequently hampered by the limited permeability of the outer membrane is considered as a frequently occurring reason for the antibiotic resistance of Gram-negative bacterial pathogens. In order to improve the transport of antibiotics into the periplasmic

space or the cytoplasm of bacteria, conjugate structures were synthesized with siderophores and antimicrobials. The idea was to use the siderophores as ‘Trojan horses’ for a facilitated penetration of antibiotics into the cells pathogenic microbes. For example, U.S. Pat. No. 6,013,647 describes benzoxazinedi one derivatives that are effective as siderophores against gram-negative bacterial strains, and conjugates of these derivatives with active ingredients such as antibiotics. It is not described to conjugate photosensitizers for anti-microbial treatments.

However, this strategy, to improve the transport of an intrinsically active antibiotic into the pathogenic microbes, and its realization by siderophore-antibiotic conjugates (see c.f. Heinisch L, Wittmann S, Stoiber T, Berg A, Ankel-Fuchs D and Möllmann U; J. Med. Chem. 2002, 45, 3032-3040; Wittmann S, Schnabelrauch M, Scherlitz-Hofmann I, Möllmann U, Ankel-Fuchs D and Heinisch L; Bioorganic&Medicinal Chemistry 10, 2002, 16-59-1670; U.S. Pat. No. 6,380,181 and 6,013,647) cannot overcome the common problem of antibiotic resistance.

Siderophores, as conjugates or included in formulations, have also been described for use in anti-cancer treatments. Examples of such therapeutic molecules described include photosensitizers. The capacity of siderophores in these anti-cancer treatments was, however, limited to enhancing the build-up of photosensitizers in cancerous tissue. Siderophores have not been used to target photosensitizers to cancerous cells, and also have not been used to target photosensitizers to microbes such as bacteria.

WO 02/094271 A1 describes a homogeneous conjugate for targeting and treating cancerous cells comprising an anti-cancer drug and a targeting protein. One described anti-cancer drug is a photosensitizer, and the preferred protein is transferrin. Because transferrin delivers protein, it is utilized as a targeting component due to the fact that cancer cells have transferrin receptors on their surfaces due to their increased need for iron. This method is restricted to anti-cancer treatments, and does not describe conjugates effective for anti-microbial therapy.

WO 02/09690 describes pharmaceutical compositions for treatment of disorders or anomalies of epithelial-lined body surface, comprising a photochemotherapeutic agent joined with a mucoadhesive agent. Optionally, a surface penetrating agent and/or one or more chelating agents may also be included. Such photochemotherapeutic agents include photosensitizers such as psoralens, porphyrins, chlorins and phthalocyanines, and precursors such as 5-aminolevulinic acid (ALA). The chelating agents may be administered in the same composition or administered after the photosensitizer-mucoadhesive composition is applied. The chelating agents, which may include some types of siderophores, may aid in promoting a build-up of protoporphyrin precursors if ALA or other precursors are included in the composition. This invention does not disclose photosensitizer-siderophore conjugates, and additionally provides that siderophores may be used only to enhance the concentration of photosensitizer precursors, and not to target or penetrate microbial cells. Lastly, this invention does not disclose the use of siderophores in anti-microbial therapy.

U.S. Patent Application No. 2002/0061871 A1 discloses pharmaceutical compositions including a protoporphyrin precursor photochemotherapeutic agent together with vascular stroma-localizing photosensitizers. The composition may also include surface penetrating agents and/or chelating agents. As with the previous publication, the chelating agents are not conjugated with the photosensitizers or precursors and additionally the formulation is not contemplated for use as an anti-microbial treatment and does not act to target microbes such as bacteria

WO 02/091991 describes homogeneous conjugates of drug molecules and protein molecules that preferentially bind to diseased cells in a predetermined molecule. The conjugate is produced by first adding linker molecules to the drug molecules and then adding the drug-linker molecule to the protein molecule. This invention does not utilize siderophores to target microbes.

U.S. Pat. No. 6,492,420 describes esters of 5-aminolevulinc acid for use in photodynamic therapy. Chelating agents such as siderophores may be included in a composition along with the ALA esters to produce a build-up of photosensitizer precursors in diseased cells. The siderophores do not act as a targeting agent and only serve to increase the photosensitive effect of photosensitizers that have been applied to the diseased tissue.

There has not yet been described a treatment utilizing photosensitizers targeted with siderophores for anti-microbial therapy. Thus, there exists a need for an anti-microbial therapy in which microbial cells can be penetrated and thus effectively and specifically destroyed, as well as a need for a treatment that is not rendered ineffective by antibiotic resistance. The present invention meets this need.

### OBJECTIVES AND BRIEF SUMMARY OF THE INVENTION

It is an object of the present invention to provide an improved anti-microbial treatment utilizing photodynamic therapy.

It is an object of the present invention to provide a photosensitizer composition with improved selectivity for pathogenic microbes such as bacteria.

Briefly stated, the present invention describes “siderophore-photosensitizer conjugates”, their synthesis and use in photodynamic antimicrobial therapy (PACT). The advantage of this method is improvement of photodynamic antimicrobial therapy against, for example, pathogenic micro-organisms such as bacteria and fungi. Naturally occurring and synthetically available siderophore structures are conjugated chemically with photoactive compounds such as Chlorin e₆ to improve their penetration into bacterial cells and to increase antibacterial efficacy of photosensitizers via microbial proteins that recognize and transport iron-loaded siderophores. In this way, photosensitizers can be transported inside bacteria that otherwise could not cross the cell wall and membranes. Photodynamic activation of photosensitizers inside the cells of pathogenic microbes enables a more effective inhibition of cellular functions than application at the outer side of the cells. The siderophore-transporting systems of microbes are known to be specific for bacteria and fungi. Consequently, siderophore conjugates with photosensitizers are not taken up by mammalian cells and photodynamic effects can thus be exerted specifically on pathogenic microbes. Applications of the present invention include highly efficient treatment of pathogenic gram-negative and -positive bacteria such as Pseudomonas aeruginosa, Escherichia coli, Streptococcus pyogenes, Staphylococcus aureus, treatment of microbial infections that often occur in chronic wounds as well as therapy of other antibiotic resistant microbial infections.

The above, and other objects, features and advantages of the present invention will become apparent from the following description read in conjunction with the accompanying figures

### BRIEF DESCRIPTION OF FIGURES

FIG. 1 illustrates an example of the synthesis of a siderophore moiety with meso-pyro-pheophorbide FIG. 2 illustrates an example of the synthesis of a siderophore moiety with hexamethylenediamine-meso-pyro-pheophorbide.

### DETAILED DESCRIPTION OF PREFERRED EMBODIMENTS

The present invention describes the synthesis and use of photosensitizer-siderophore conjugates for site-specific photosensitizer transport to microbial bodies such as bacteria and fungi. Covalent binding of siderophore structures to photosensitizers is a new way to produce new chemical structures that act as a shuttle for the active transport of photoactive molecules into bacterial cells and for photodynamic antibacterial therapy.

Conjugate structures of photoactive dyes are disclosed that possess the general formula I:

A−B I

wherein A represents photoactive dyes such as erythrosine B, chlorin e₆ and pheophorbide a, and B means a siderophore-type chelator of trivalent iron ions containing catecholate or hydroxamate structures. Furthermore, chemical procedures for the preparation of compounds of the general formula I are disclosed, characterized by chemical couplings of reactive groups of photoactive dyes such as hydroxyl, amine or carboxyl with reactive substituents of siderophore-type chelators of ferric ions.

In a preferred embodiment, conjugate structures of the general formula I are provided where the siderophore represented by B is in the form of either of the substructures represented by X and Y: wherein n=1 to 6, and Z=CO or NH

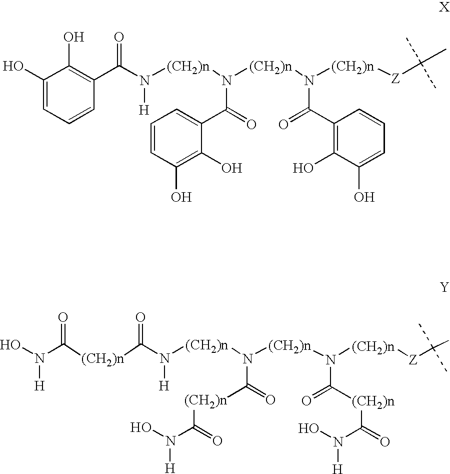

where X is a catecholate type siderophore and Y is a hydroxamate type siderophore, and wherein n=1 to 6, and z=CO or NH.

Moreover, the invention concerns the preparation of therapeutically useful formulations and its usage in the photodynamic therapy of infectious diseases caused by bacteria or fungi.

In another preferred embodiment of the present invention, the photosensitizer, represented by A, connected to the siderophore-type molecule of type X or Y is a chlorin or bacteriochlorin-type photosensitizer derived from chlorophyll or bacteriochlorophyll. Siderophore-photosensitizer conjugates of this type are easily prepared by reacting a siderophore of type X or V possessing a free amino group with a chlorin-type or bacteriochlorin-type photosensitizer possessing a free carboxyl group using conventional peptide bond coupling chemistry (e.g. anhydride method, active ester method).

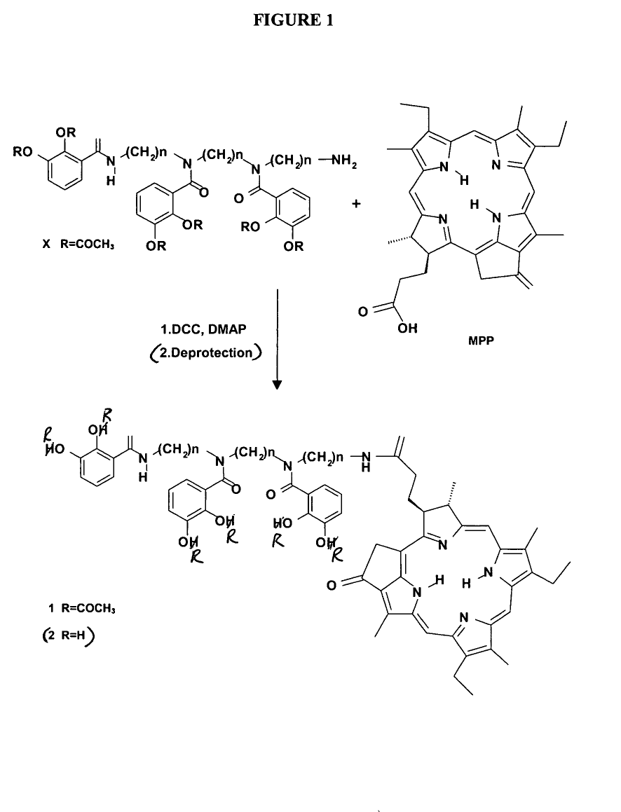

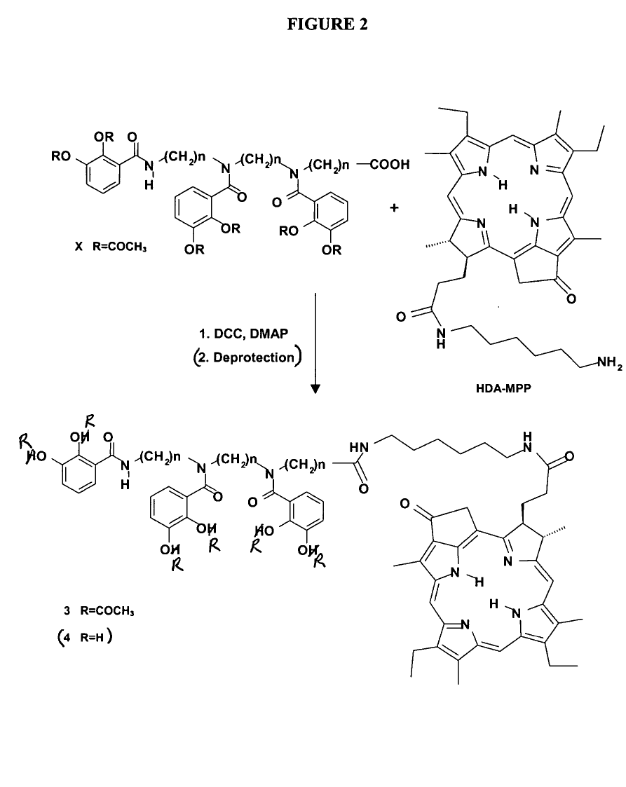

In yet another preferred embodiment, a compound consisting of a catecholate-type siderophore as represented by X is connected to meso-pyro-pheophorbide (MPP), which is prepared, for example, as indicated in FIG. 1. In this example, z=NH.

[image]

The present invention is further illustrated by the following examples, but is not limited thereby.

### EXAMPLE 1

As shown in FIG. 1, compounds 1 and 2 can be obtained in good yields by coupling a compound X protected by acetyl residues at the phenol hydroxyls with a photosensitizer such as meso-pyro-pheophorbide (MPP) containing a carboxyl function. Specifically, in this example, formation of a conjugate formed by X with R═COCH₃ and MPP can be furnished following the subsequent protocol:

Equimolar amounts of X as shown in FIG. 1, with R═COCH₃, and MPP are used for preparation of compounds 1 or 2 . 10 mmoles of X were dissolved under stirring at room temperature in 100 ml of dry chloroform containing 50 mg of N,N-dimethylaminopyridine (DMAP).

Subsequently 10 mmoles of MPP were added. Stirring was continued until a homogenous solution was formed. To accomplish formation of an amide bond between X and MPP, a solution of 30 mmoles Dicyclohexylcarbodiimide (DCC) in 50 μm CHCl₃ was added dropwise within 60 min to the stirred solution of educts. Thereafter, stirring was continued for 5 hours whereby the temperature was increased to 50° C. The mixture was cooled to ambient temperature, and the precipitated dicyclohexylurea was removed by filtration.

The chloroform filtrate was evaporated in vacuo and 2 portions of the residue were dissolved in 50 ml methanol. The solution was chromatographed on Sephadex LH-20 (column 10 cm×100 cm, methanol as eluent) whereby the conjugate structure 1 composed of X(R═COCH₃) and MPP was first eluted due to its higher molecular weight. The fractions containing 1 from several chromatographic separations were combined to yield 10 g (80% yield).

The conjugate 1 thus obtained can be used as such in photodynamic therapy of bacterial infections due to the proved efficiency of X(R═COCH₃) as a siderophore.

Alternatively, conjugate 1 can be deacetylated under moderately acidic conditions to yield 2. Thus 1 g of 1 was dissolved in 200 ml methanol containing 1.8 g oxalic acid (1M solution) and was refluxed for 2 hours. Thereby the phenol esters were saponificated but the amide bonds remained stable. Subsequently the solvent was evaporated in vacuo and residue was chromatographed on Sephadex LH-20 (column 10 cm×100 cm, methanol as solvent). First the catechol type Siderophore conjugate 2 with MPP was eluted and separated by this way from oxalic acid. Yield 0,8 g (80%).

### EXAMPLE 2

Structures 3 and 4 as shown in FIG. 2 can be obtained by coupling the carboxylic group of catechol type siderophore X(R═COCH₃) with meso-pyro-pheophorbide substituted by a diamine residue such as hexamethylene diamine (HDA-MPP).

The procedure for coupling X with HDA-MPP and the subsequent purification by chromatography on Sephadex LH-20, is identical to the procedure described in example 1.

By that method, conjugate 3 was obtained in 80% yield. By the same procedure as described in example 1, conjugate 3 was converted to the deacetylated catechol 4 in 80% yield.

[image]

Physico-Chemical Properties of Conjugates 1, 2, 3 and 4:

Appearance:

bluish-violet, solid (1, 2, 3 and 4)

Solubility:

1 and 3: alcohols (MeOH, EtOH,)CHCl₃, DMSO

2 and 4: alcohols (MeOH, EtOH, ProOH), DMSO

Molecular weight:

1: M=1324, C₇₆H₉₂N₈O₁₃

2: M=1240, C₇₂H₈₈N₈O₁₁

3: M=1437, C₈₂H₁₀₀N₉O₁₄

4: M=1353, C₇₈H₉₉N₉O₁₂

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A molecular conjugate represented by formula 1:
I=A−B
wherein A is at least one photosensitizer moiety and B is at least one siderophore that selectively attaches to receptor sites on a targeted microbe.

2. The molecular conjugate according to claim 1, wherein said at least one photosensitizer is selected from the group consisting of porphyrins and active derivatives thereof, phtalocyanins, metallo derivatives thereof, dyes, and synthetic photosensitizers.

3. The molecular conjugate according to claim 1, wherein said at least one photosensitizer is at least one photoactive dye selected from the group consisting of erythrosine B, chlorin e₆ or pheophorbide a, and B is a siderophore-type chelator of trivalent iron ions containing catecholate or hydroxamate structures.

4. A method for the preparation of the conjugates of claim 1, comprising the step of chemical coupling of reactive groups of photoactive dyes with reactive substituents of siderophore-type chelators of ferric ions.

5. The method according to claim 4, wherein said chemical coupling step is accomplished through reactive groups of said photoactive dyes, which are selected from the group consisting of a hydroxyl group, an amino group, and a carboxyl group.

6. The molecular conjugate according to claim 1, wherein B is selected from the group consisting of X and Y: 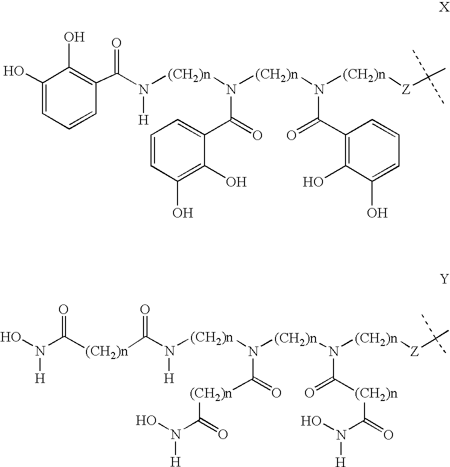
wherein X is a catecholate type siderophore and Y is a hydroxamate type siderophore, and wherein n=1 to 6, and Z is selected from the group consisting of CO and NH.

7. Use of the molecular conjugate of claim 1 in photodynamic therapy of infectious diseases caused by microbes, comprising the steps of:
a. administering to a host organism a pharmaceutically effective amount of said molecular conjugate of claim 1, and
b. irradiating said host organism with a preselected wavelength to cause said photosensitizer to produce a cytotoxic effect on said microbes.

8. The use of said molecular conjugates according to claim 7, wherein said microbes are selected from the group consisting of bacteria and fungi.

9. Usage of compound of the general formula I in therapeutically useful formulations.
